(19) United States
(12) Patent Application Publication
(10) Pub. No.: US 2005/0192603 A1
(43) Pub. Date: Sep. 1, 2005

(54) Extravascular anastomotic components and methods for forming magnetic anastomoses

(75) Inventors: David Cole, San Mateo, CA (US); Michael Reo, Redwood City, CA (US); Dean Carson, Mountain View, CA (US); Darin Gittings, Sunnyvale, CA (US); A. Adam Sharkawy, Redwood City, CA (US); Mark Foley, Menlo Park, CA (US)

Correspondence Address: HOEKENDIJK & LYNCH, LLP, P.O. BOX 4787, BURLINGAME, CA, 94011-4787, US

(73) Assignee: MEDTRONIC AVECOR CARDIOVASCULAR, INC. a Minnesota corporation, Minneapolis, MN (US)

(21) Appl. No.: 11/039,705

(22) Filed: Jan. 19, 2005

Related U.S. Application Data
(63) Continuation of application No. 10/022,187, filed on Dec. 13, 2001, now abandoned.
(60) Provisional application No. 60/255,635, filed on Dec. 13, 2000.
(60) Provisional application No. 60/323,923, filed on Sep. 15, 2001.

(30) Foreign Application Priority Data
Aug. 10, 2001 (WO) PCT/US01/25197

(51) Int. Cl. A61B 17/08

(52) U.S. Cl. 606/153

(57) ABSTRACT

Methods and devices using magnetic force to form an anastomosis between hollow bodies. End-to-side, side-to-side and end-to-end anastomoses can be created without using suture or any other type of mechanical fasteners, although such attachment means may be used in practicing some aspects of the invention. Magnetic anastomotic components may be attached to the exterior of a vessel, e.g., by adhesive, without extending into the vessel lumen. Various magnetic component configurations are provided and may have different characteristics, for example, the ability to match the vessel curvature or to frictionally engage the vessel.

### CROSS-REFERENCE TO RELATED APPLICATIONS

The present application claims priority from the following copending patent applications: application Ser. No. 09/915,226, filed Jul. 23, 2001; application Ser. No. 09/638,805, filed Aug. 12, 2000; application Ser. No. 09/562,599, filed Apr. 29, 2000; provisional application Ser. No. 60/255,635, filed Dec. 13, 2000; application Ser. No. 09/851,400, filed May 7, 2001; provisional application Ser. No. 60/323,923, filed Sep. 15, 2001 and PCT application no. PCT/US01/25197 filed Aug. 10, 2001. The entire disclosure of each of the above-referenced patent applications is expressly incorporated herein by reference.

### BACKGROUND OF THE INVENTION

1. Field of the Invention

The invention relates to forming anastomoses between two hollow bodies, and more specifically, using magnetic force to form such anastomoses.

2. Description of Related Art

Various non-suture based anastomotic systems have been proposed in the art, however, none has performed well enough to receive any significant level of acceptance in the field. Many of the proposed couplings fail to remain sufficiently patent, either acutely or chronically. Another technical challenge is to create an anastomosis that produces a fluid-tight seal between the hollow bodies. This is due in large part to the difficulty in securing an anastomotic component without overly traumatizing the tissue and without placing too much foreign material in the vessel lumen.

### SUMMARY OF THE INVENTION

One embodiment of the invention provides an anastomotic component that is coupled or attached to the wall of a vessel without protruding into the lumen of the vessel or penetrating the vessel wall. That is, substantially none of the anastomotic component or assembly is located within the vessel lumen (i.e., after the anastomosis has been formed). As a result, there is preferably no foreign structure or material disposed within the target vessel lumen after creating the anastomosis.

The specific manner in which the anastomotic component is secured to the vessel may vary according to the invention. In one embodiment biocompatible adhesive is used to secure a component to the exterior of the target vessel wall without extending into the lumen. This component is coupled to a magnetic or ferromagnetic assembly carried on a graft vessel. Another embodiment uses adhesive to secure the anastomotic components to both vessels.

According to further embodiments magnetic force is used in combination with an additional locking force, for example, a mechanical connection, to maintain the vessels in proper position and provide heightened resistance to pressure fluctuations that might occur post-formation of the anastomosis. Alternative constructions for the anastomotic components are disclosed, as are various delivery devices and methods for deploying the components.

### BRIEF DESCRIPTION OF THE DRAWING FIGURES

Other features, benefits and advantages of the present invention will be apparent from the following detailed description of preferred embodiments thereof taken in conjunction with the accompanying drawing figures, wherein:

FIGS. 1A and 1B are, respectively, plan and elevation views of a magnetic anastomotic component constructed according to one embodiment of the invention;

FIG. 1C is an elevation view of a magnetic anastomotic component constructed according to an alternative embodiment of the invention;

FIGS. 2A and 2B are, respectively, perspective and elevation views of a magnetic anastomotic component constructed according to another embodiment of the invention;

FIGS. 3A and 3B are, respectively, perspective and elevation views of a magnetic anastomotic component constructed according to yet another embodiment of the invention;

FIG. 4 is a perspective view showing the anastomotic component of FIGS. 3A and 3B attached to a vessel;

FIGS. 5A and 5B are perspective views showing an anastomotic component being secured to a vessel according to another embodiment of the invention;

FIGS. 6A-6C are elevation views showing anastomotic components constructed according to different embodiments of the invention being used to form an anastomosis between two vessels;

FIGS. 7A-7C are elevation views showing an anastomotic component being secured to a vessel according to another embodiment of the invention;

FIG. 7D is an elevation view showing the component of FIGS. 7A-7C being secured to an end of a vessel;

FIGS. 8A and 8B are perspective views of magnetic anastomotic components provided with tissue anchoring elements according to another embodiment of the invention;

FIGS. 9A-9C are elevation views, in section, showing magnetic anastomotic components provided with tissue traction-enhancing structure according to another embodiment of the invention;

FIGS. 10A-10C are, respectively, perspective views and a sectional view of one of the magnetic anastomotic components shown in FIG. 9C;

FIGS. 11A and 11B are, respectively, plan and sectional views of a magnetic anastomotic component provided with tissue gripping structure according to another embodiment of the invention;

FIG. 11C is a sectional view of an anastomotic component having an alternative tissue gripping structure;

FIGS. 12A-12D are, respectively, perspective, side elevation, end elevation and plan views of a magnetic anastomotic component constructed according to another embodiment of the invention;

FIGS. 13A-13C are, respectively, perspective, side elevation and end elevation views of an anastomosis formed by a pair of magnetic anastomotic components constructed according to another embodiment of the invention;

FIGS. 14A-14D are, respectively, plan, perspective, end elevation and side elevation views of a magnetic anastomotic component constructed according to another embodiment of the invention;

FIGS. 15A-15D are, respectively, plan, perspective, end elevation and side elevation views of a magnetic anastomotic component having a similar construction as the component shown in FIGS. 14A-14D;

FIGS. 16A-16B perspective views showing an anastomotic component being mounted to the exterior surface of a hollow body according to one embodiment of the invention;

FIGS. 16C-16D perspective views showing an anastomotic component being mounted to the exterior surface of a hollow body according to one embodiment of the invention;

FIGS. 17A and 17B are, respectively, perspective and end elevation views of an extravascular anastomosis created according to one embodiment of the invention;

FIGS. 18A-18D are perspective views showing an anastomotic component being mounted to the exterior surface of a hollow body according to another embodiment of the invention; and

FIG. 19 is an end elevation view of a magnetic anastomotic component mounted to the exterior of a vessel according to one embodiment of the invention.

### DETAILED DESCRIPTION OF PREFERRED EMBODIMENTS

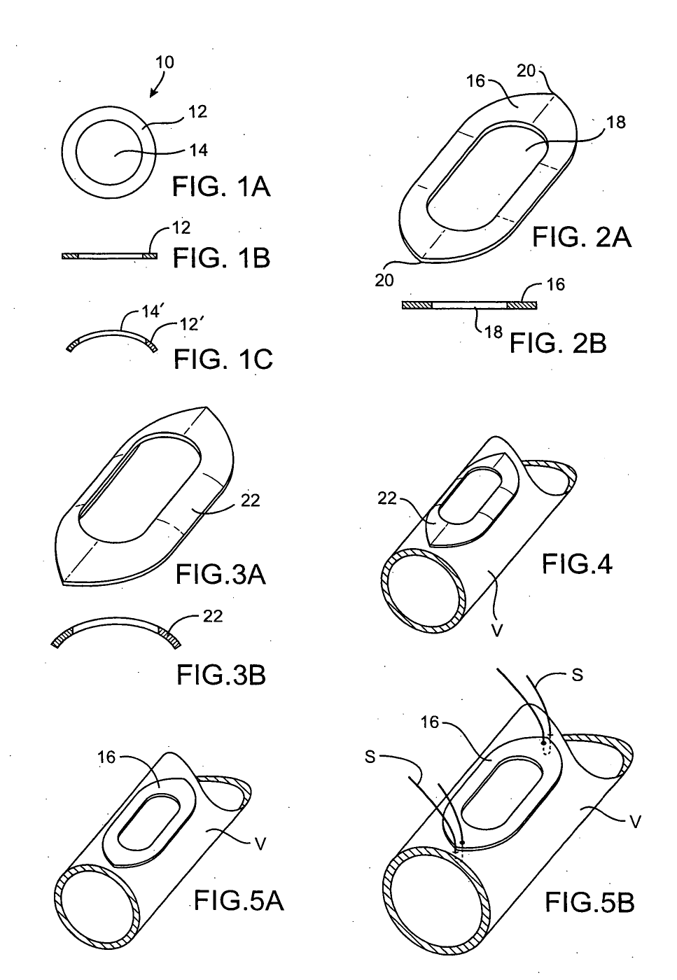

FIGS. 1A and 1B show a first embodiment of a magnetic anastomotic component 10 having a ring-shaped body 12 and an opening 14. As shown in FIG. 1B the component body 12 is generally flat. However, as shown in FIG. 1C, the body 12 may be curved, for example, to match the curvature of a vessel to which it is secured.

FIG. 2A shows a magnetic anastomotic component 16 with an opening 18. The body of the component 16 has an oval or elliptical shape with leading edges 20 for facilitating atraumatic introduction into a vessel. As shown in FIG. 2B, the component 16 is flat. As in the above embodiment, however, the component 16 could be curved instead, for example, in a manner similar to the curvature of the anastomotic component 22 shown in FIGS. 3A-3B. Also, the curvature may extend over all or a portion of the length (or width) of the component.

FIG. 4 shows the anastomotic component 22 of FIGS. 3A and 3B mounted on the side wall of a vessel V. According to the preferred embodiments, the component is secured to the vessel without projecting into the vessel lumen, thereby avoiding potential problems associated with foreign material located in the vessel lumen. The component may be secured to the exterior of the vessel by suitable means, for example, adhesive, mechanical fasteners, or both.

FIGS. 5A and 5B show the anastomotic component 16 of FIGS. 2A and 2B mounted on a side wall of a vessel V. FIG. 5B shows mechanical fastening means, the illustrated means being in the form of sutures S, which are used to attach the component 16 to the vessel V. While sutures S are shown, it will be recognized that any suitable mechanical fastener may be used, e.g., clips, stents, barbs, hooks, wires, etc.

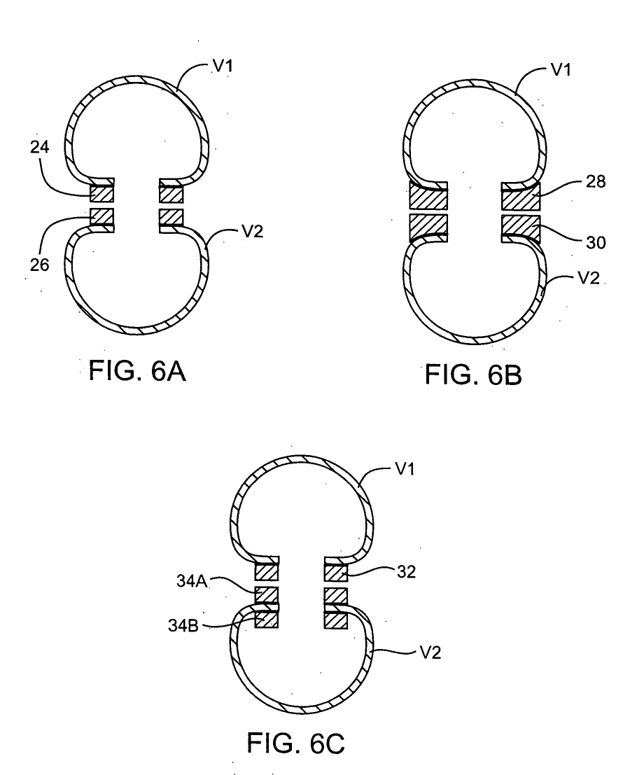

In the embodiments of FIG. 4 and FIGS. 5A-5B, the anastomotic component is secured to the exterior of the vessel wall by suitable means. FIGS. 6A and 6B show anastomoses between two vessels V1 and V2. In FIG. 6A, the vessels have mounted thereto, respectively, magnetically attracted anastomotic components 24, 26. (For clarity, the components are shown slightly separated.) The components 24, 26 are rectangular in cross-section. In FIG. 6B the vessels V1, V2 have mounted thereto, respectively, components 28, 30. The components 28, 30 are provided with a curved exterior surface that generally corresponds to the curvature of the walls of vessels V1 and V2.

The anastomoses shown in FIGS. 6A and 6B are created without placing any component portion in the vessel lumen. FIG. 6C shows an embodiment wherein a vessel V1 has an anastomotic component 32 secured thereto, while a vessel V2 has an anastomotic component comprising portions 34A and 34B secured thereto. Unlike the embodiments of FIGS. 6A and 6B, thought, the portion 34B of the one component is disposed within the lumen of vessel V2.

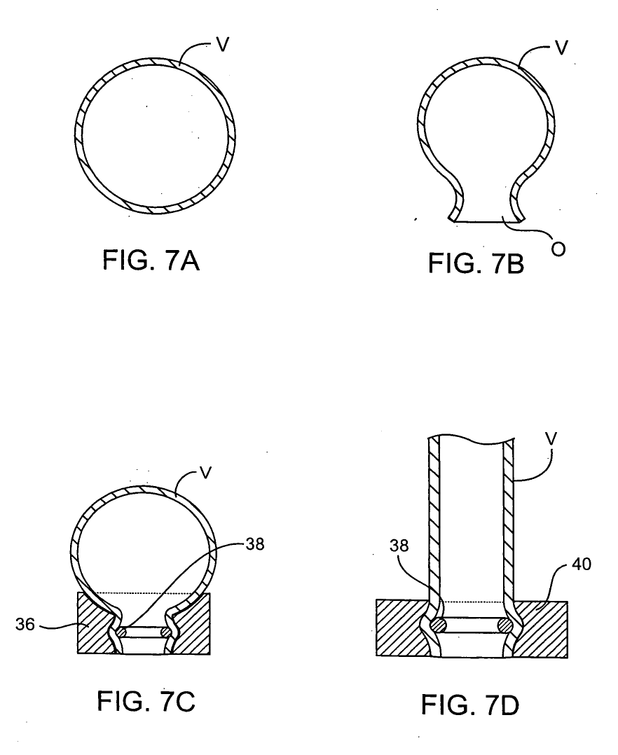

FIG. 7A shows a vessel V prior to forming an opening in the wall thereof. FIG. 7B shows the vessel V after an opening O has been formed therein. FIG. 7C shows an anastomotic component 36 positioned around the outside of the opening in the vessel V. An internal locking member 38, which may be in the form of a snap ring, is positioned within the vessel lumen and cooperates with a groove in component 36 to secure the vessel and component together. FIG. 7D shows an anastomotic component 40 positioned around the end of a vessel V. The internal locking component 38 cooperates with a groove in the component 40 to secure the component to the end of the vessel (as opposed to the side wall of the vessel, as in the previous embodiments).

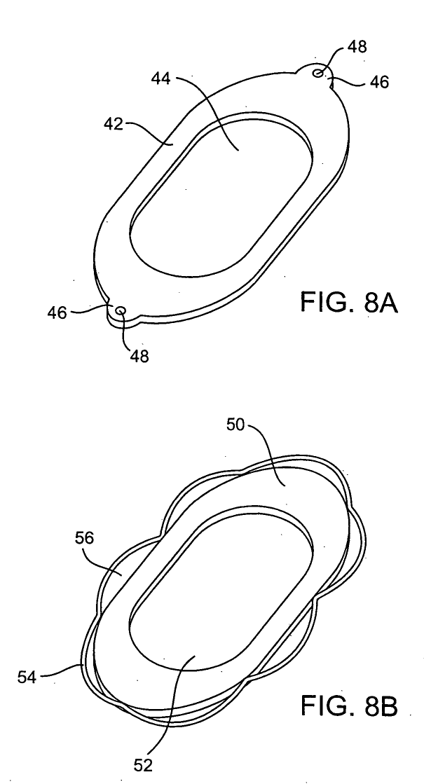

FIG. 8A shows a magnetic anastomotic component 42 having an opening 44 and a pair of attachment tabs 46 with openings 48. The component 42 is mounted to the exterior of a vessel (not shown), for example, by passing a fastener (also not shown) through each opening 48 into engagement with the vessel tissue. Alternatively, the tabs 46 and openings 48 may be used as secondary securing means, for example, if the component 42 is secured to the vessel by other means, e.g., adhesive.

FIG. 8B shows a magnetic anastomotic component 50 having an opening 52 and attachment structure 54 to facilitate securing the component to a vessel (not shown). As above, the structure 54 may be used alone or in combination with other means for securing the component to the vessel. In the illustrated embodiment, the attachment structure 54 is affixed to the component 50 to define a plurality of openings 56 which may be use to receive sutures, clips, clamps, pins, barbs, or other securing or fastening means.

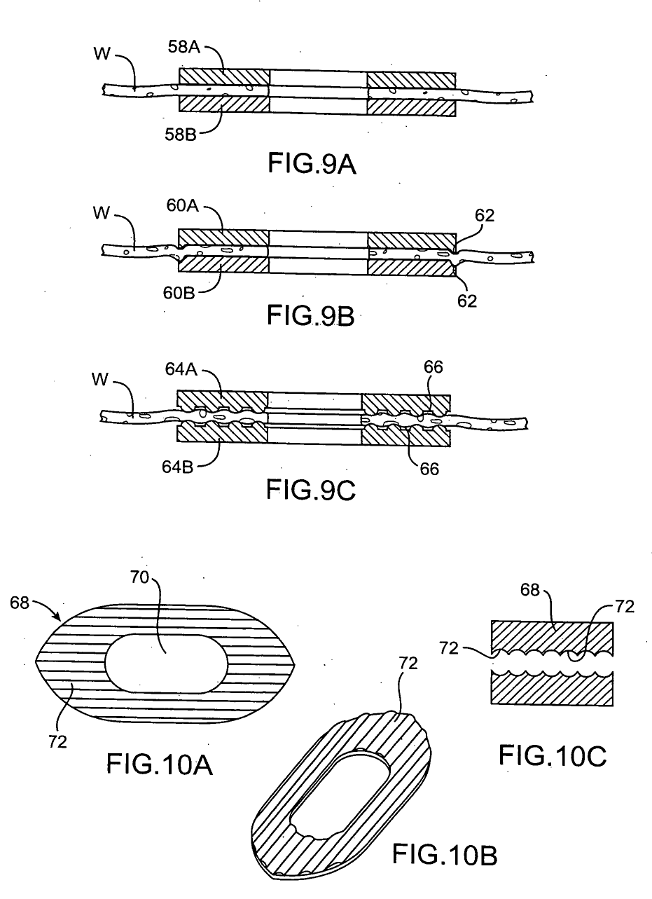

One benefit of the embodiments of FIGS. 8A-8B and 9A-9B is that the attachment structure is disposed away from (or below) the magnetic coupling surface of the component. That is, the exposed surface of the first component is free to mate with the exposed surface of the second component without interference from the attachment structure. As a result, one or both components can be firmly affixed to its vessel without adversely affecting the anastomosis.

FIGS. 9A-9C show three embodiments of magnetic anastomotic components that are provided with structure for increasing the traction or gripping force between the components and a vessel to which they are secured. In FIG. 9A, anastomotic component portions 58A, 58B sandwich a vessel wall W and are preferably provided with a layer of material to enhance engagement with the tissue. FIG. 9B shows component portions 60A and 60B, each of which includes a projection 62 at the end thereof which grabs the tissue of the vessel wall W, thereby enhancing securement. FIG. 9C shows anastomotic component portions 64A and 64B, each of which is provided with a series of grooves or annulations 66 that grippingly engage the tissue of the vessel wall W.

FIGS. 10A-10C show an anastomotic component 68 with an opening 70 and a plurality of grooves or bumps 72. The grooves or bumps 72, which may also be in the forms of ridges, serrations, sharp or dull edges, etc., grab the tissue of the vessel to which the component is secured, which provides additional attachment force. FIG. 10C shows the ridges 72 having sharp points 74 to further enhance engagement with the tissue.

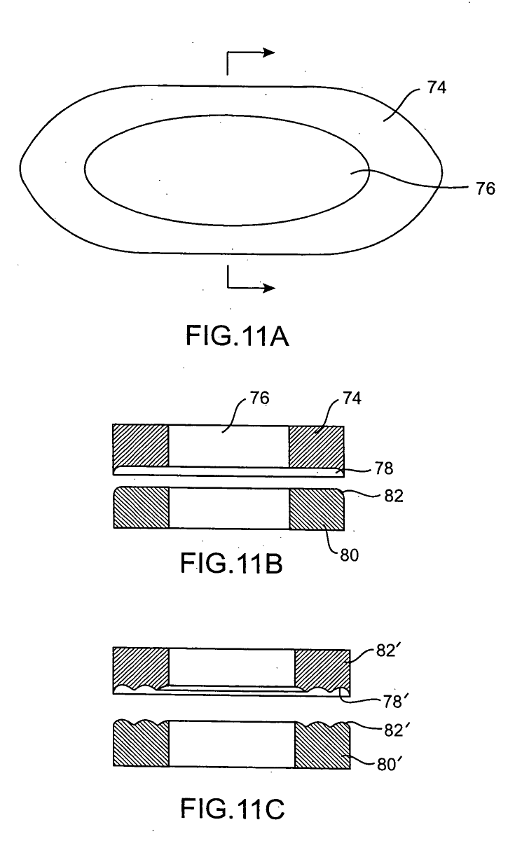

FIGS. 11A-11C show a magnetic anastomotic component 74 with an opening 76 and a peripheral edge 76 that defines a sharp point 78. As shown in FIG. 11B, a second anastomotic component 80 may be used with the component 74, the component 80 having a complimentary-shaped edge 82 which cooperates with the edge 76 to sealingly and grippingly grab tissue of a vessel to which the components are secured. FIG. 11C shows a variation of the component 74 wherein a plurality of edges 74′ and 76′ are provided. A modified second component 80′ has a plurality of complimentary edges 82′ that mate with the edges 76′ of component 74′. In each of these embodiments the force-increasing structure is shown running along the entire length of the component. It will be appreciated that such structure may be extend along all or any portion of the component, and could extend across the width or longitudinal axis of the component, rather than along the axis, as in FIGS. 10A-10C.

The attachment force-increasing embodiments of FIGS. 9A-9C, 10A-10C and 11A-11C provide several benefits. In addition to enhancing attachment of the component to the vessel, the resulting anastomosis may have higher resistance to bursting under high pressures, e.g., acute pressure increases. For example, placing a rough or bumpy parylene coating on the surface of a magnetic component produces higher burst pressure resistance than using a smooth surface. It is desirable to increase pressure resistance, preferably without increasing the risk of occlusion.

According to the invention, the components described above may be secured to the vessel by various means. For example, the component may be adhesively attached to the exertion of the vessel so that the lumen of the vessel is free of any component portion. In addition to the adhesive securement of the component, any of the above-described traction or tissue-gripping structure may be used as well. Additionally, the component may be provided with tabs or other attachment structure as described above.

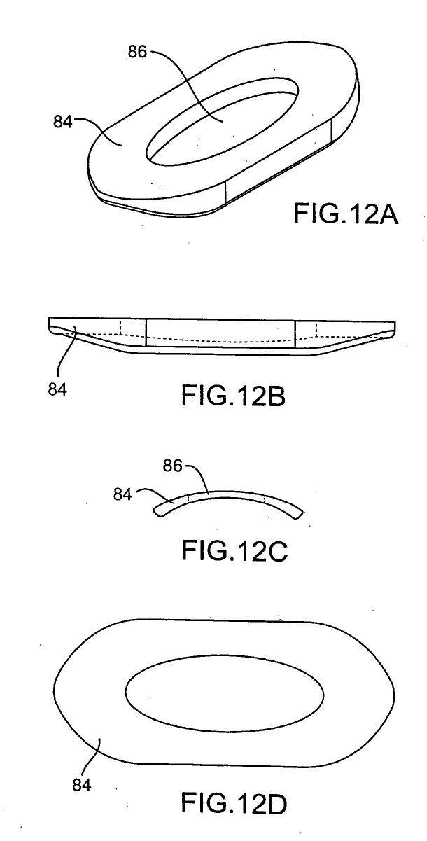

FIGS. 12A-12D show a magnetic anastomotic component 84 having a rounded configuration designed to mate with the curvature of a vessel, and an opening 86 adapted to communicate with the vessel lumen. The thickness of the component 84 is tapered across its width (FIG. 12C) and may be tapered more or less from the specific configuration shown.

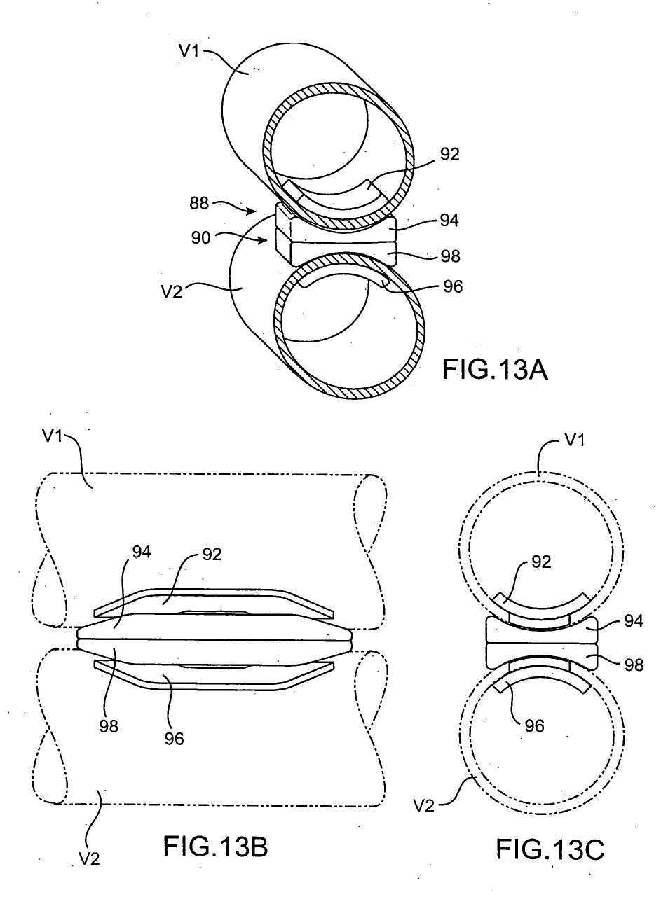

FIGS. 13A-13C show an anastomosis created according to another embodiment of the invention. A first vessel V1 and a second vessel V2 are provided with respective magnetically-attracted components 88, 90. The component 88 has an intravascular portion 92 and an extravascular portion 94, while the component 90 has an intravascular portion 96 and an extravascular portion 98 as shown best in FIGS. 13A and 13C. The extravascular portions 94, 98 of the respective components are flat and provide a flat engagement to enhance the magnetic force holding the components together.

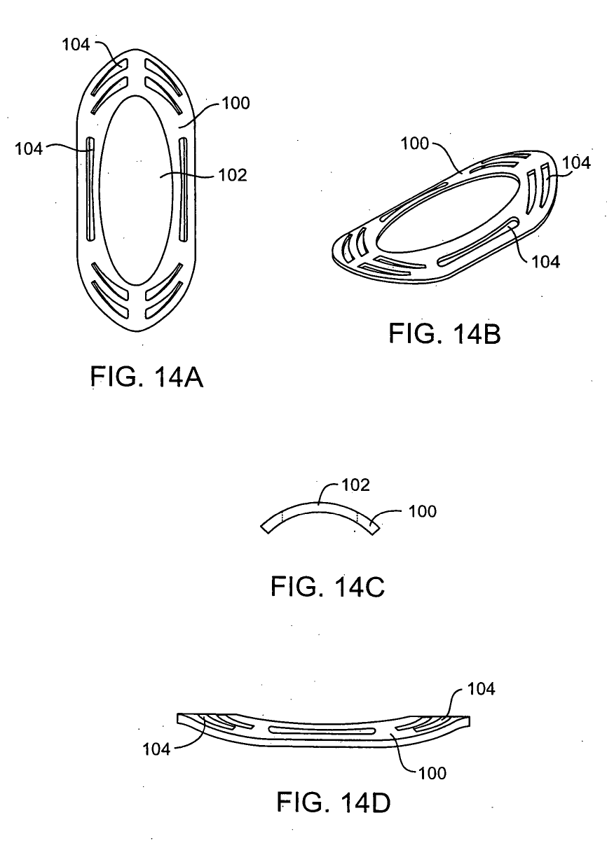

FIGS. 14A-14D show a magnetic anastomotic component 100 having a luminal opening 102 and a plurality of slots 104. The slots 104 serve any of several purposes including allowing tissue ingrowth to promote attachment to the vessel, enhance traction between the component 100 and the vessel to which it is attached, etc.

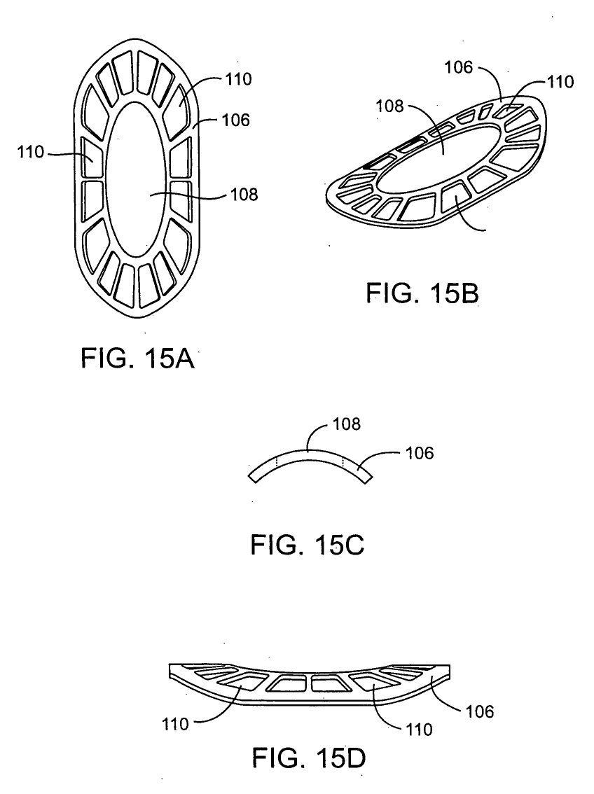

FIGS. 15A-15D show a magnetic anastomotic component 106 with a luminal opening 108 and a plurality of apertures 110 disposed around its perimeter. The apertures 110 give the component 106 a frame-like structure and may serve any of the purposes described above with respect to the previous embodiment. It will be noted that the components 106 and 100, while illustrated as being curved to match the curvature of a vessel or mating component (not shown), they may instead be flat or otherwise configured.

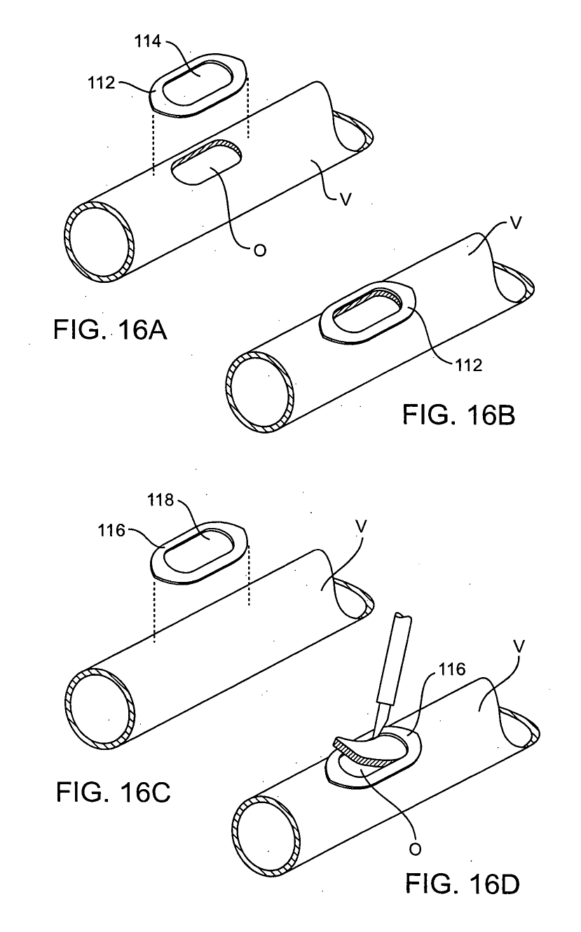

FIGS. 16A and 16B show a magnetic anastomotic component 112 being attached to a vessel V according to one embodiment of the inventions. An opening O is formed in an opening of a side wall of the vessel V and a magnetic anastomotic component 112 is moved into position such that the luminal 114 of the component is aligned with the opening O (FIG. 16B).

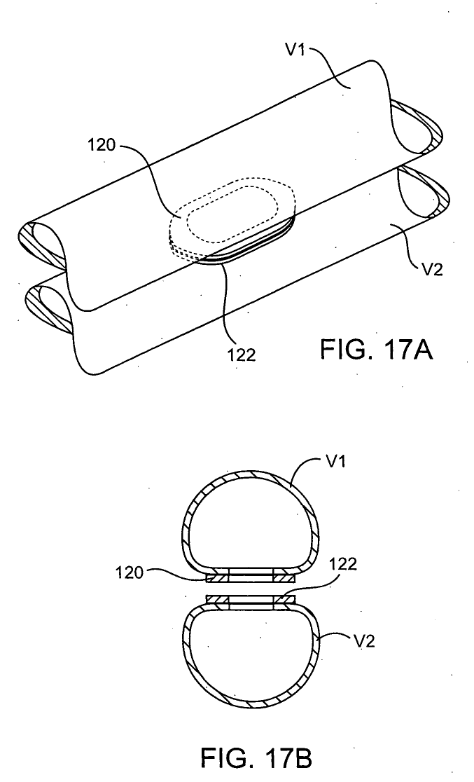

FIGS. 16C and 16D show a magnetic anastomotic component 116 being secured to a vessel V according to another embodiment of the invention. In this embodiment, the component 116 is lowered against and secured to the vessel wall as in the above embodiment. However, an opening is formed in the vessel after placing the component in this embodiment. As shown in FIG. 17B, a suitable instrument is used to remove the tissue circumscribed by the opening 116 of the component 116. The components 112 and 116 may be secured to the exterior of the wall of vessel V by any suitable means disclosed herein.

FIG. 17A shows an anastomosis between first and second vessels V1 and V2 which are provided, respectively, with magnetically attracted components 120, 122. As shown in FIG. 17B, of the components 120, 122 have mating surfaces which are positioned against each other and held by magnetism to create the anastomosis.

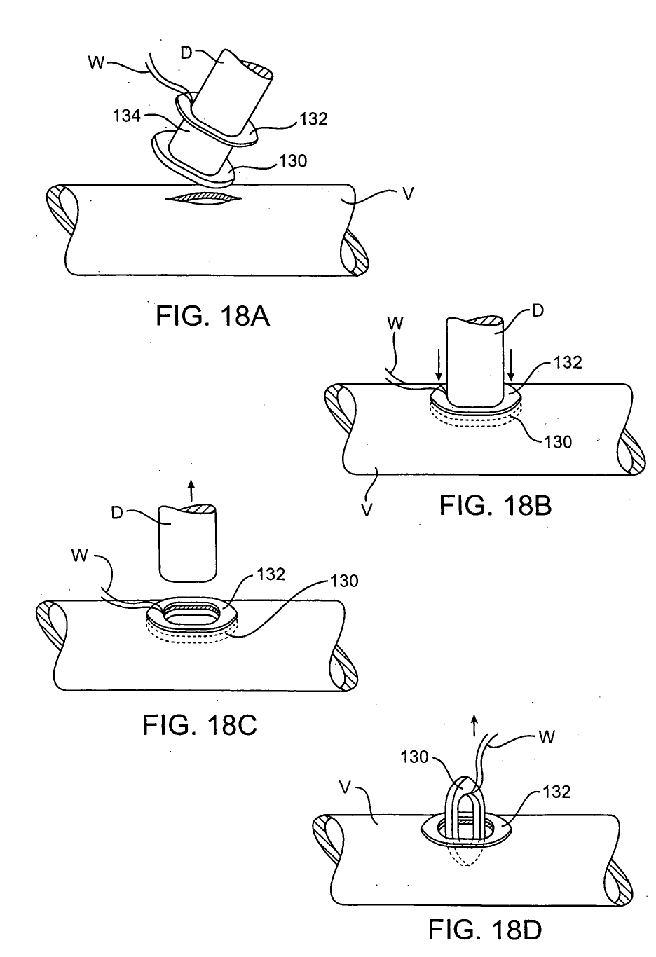

FIGS. 18A-18D show a magnetic anastomotic component being secured to the exterior of a vessel wall according to still another embodiment of the invention. Delivery device D includes an internal placement member 130 which is used to place a magnetic anastomotic component 132. The placement member 130 is positioned within the lumen of the vessel through an incision in the wall, and the anastomotic component 132 is slid down against the exterior of the vessel. Magnetic attraction holds the component 132 in position around the incision.

It should be noted that in positioning the placement member within the lumen of the vessel v, the delivery device is manipulated, typically by pulling up to tension the vessel wall, and the edges of the incision are positioned around a portion 134 of the delivery device D so as to make the incision the desired size. When the edges of the incision are so positioned, the anastomotic component 132 is slid down and the magnetic attraction captures the edges of the incision, thereby maintaining a suitable size opening.

Next, the delivery device D is removed as shown in FIG. 18C. Finally, as shown in FIG. 18D, the internal placement member 130 is pushed distally and rotated and then removed (for example, by wires W) through the incision in the vessel V. The magnetic anastomotic component 132 is preferably provided with adhesive to secure the component to the vessel. Alternatively, adhesive may be applied around the incision on the vessel and the component 132 moved into contact therewith.

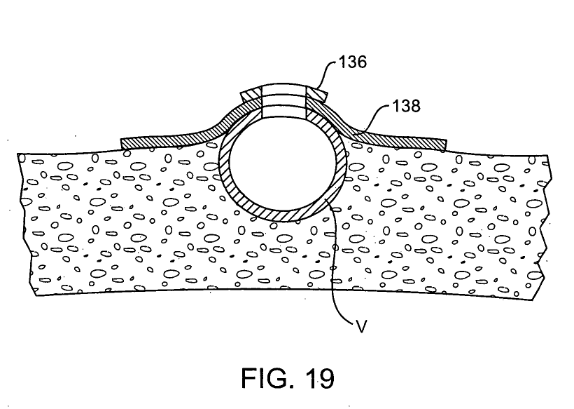

FIG. 19 shows an embodiment of the invention where a magnetic anastomotic component 136 is secured to an intermediate member 138, for example a dacron blanket, which itself is secured to the wall of a vessel V. These embodiments may be practiced by forming a blanket or surface of adhesive on the vessel exterior, and then forming the incision through the adhesive (which may be less difficult than incising the vessel wall directly).

The invention may be practiced using any suitable biocompatible adhesives. In general, fibrin sealants and cyanoacrylate esters are the two types of adhesives widely used for biological bonding. Gelatin-resorcinol-formaldehyde glues have limited use as well. Other possible bioadhesives include gelatin-resorcinol-formaldehyde glue, bovine albumin, glutaraldehyde, marine organism (mussel) based, collagen and thrombin.

Fibrin sealants are biodegradable, adhere well to connective tissue, promote wound healing, and generally have less bond strength than cyanoacrylate esters. A two-part system may be used to apply the sealant, or a one-part, ready-to-use formulation may be used instead. The adhesives used may have or not have antifibrinolytic agents (e.g., aprotinin, etc.)

Those skilled in the art will recognize that many modifications, alterations and variations of the illustrated embodiments may be made without departing from the scope and spirit of the invention as defined by the appended claims. For example, while the embodiments are described in connection with magnetic anastomotic components, it will be appreciated that various features of the invention may be practiced in conjunction with non-magnetic anastomotic components. Further, it will be appreciated that, independent of the specific illustrated embodiments, the components disclosed herein may be used to create end-to-end, end-to-side or side-to-side anastomoses, between blood vessels or any hollow anatomical structures.

1. An anastomosis device comprising:
a component configured to be secured to a vessel and having an opening adapted to be placed in fluid communication with a lumen of the vessel;
wherein the component comprises a material having the ability to produce or be attracted by a magnetic field; and
wherein the component is configured to be secured to the vessel substantially without any fixation structure being present in the vessel lumen.

2. The device of claim 1, wherein the component is configured to be secured to the vessel without any fixation structure being present in the vessel lumen.

3. The device of claim 1, wherein the component has a surface configured to be secured to the vessel wall by adhesive.

4. An anastomosis device comprising:
a component having a portion configured to be adhered to a wall of a vessel by biocompatible adhesive to define a blood flow path into the vessel; and
wherein the component is configured to be secured to the vessel wall by an additional, nonadhesive-based attachment mechanism.

5. The device of claim 4, wherein the portion of the component and the attachment mechanism are configured to secure the component to the vessel without any fixation structure being present in the vessel lumen.

6. The device of claim 4, wherein the component comprises a material having the ability to produce or be attracted by a magnetic field.

7. An anastomosis device comprising:
a component configured to be secured to a vessel and having an opening adapted to be placed in fluid communication with a lumen of the vessel;
wherein the component comprises a material having the ability to produce or be attracted by a magnetic field; and
wherein the component has a portion that is at least partially curved.

8. The device of claim 7, wherein the component is configured to be secured to the vessel wall by an adhesive.

9. A method for forming an anastomosis comprising:
(a) providing a first vessel with a first anastomotic component;
(b) providing a second vessel with a second anastomotic component; and
(c) coupling the first and second anastomotic components to place their lumens in fluid communication;
(d) wherein at least one of steps (a) and (b) is performed at least in part by securing the anastomotic component to the vessel using adhesive.

10. The method of claim 9, wherein step (c) is performed at least in part by using magnetic force to couple the anastomotic components.
